(19) United States
(12) Patent Application Publication
(10) Pub. No.: US 2003/0236483 A1
(43) Pub. Date: Dec. 25, 2003

(54) Dual drainage ocular shunt for glaucoma

(76) Inventor: David H. Ren, Dallas, TX (US)

Correspondence Address: DONALD W. MEEKER, PATENT AGENT, 924 EAST OCEAN FRONT #E, NEWPORT BEACH, CA, 92661, US

(21) Appl. No.: 10/183,781

(22) Filed: Jun. 25, 2002

(51) Int. Cl. A61M 5/00; A61F 9/00

(52) U.S. Cl. 604/8; 606/107

(57) ABSTRACT

A “cross-shaped” tubular structure shunts aqueous humor from the anterior chamber of the eye to control intraocular pressure (IOP) associated with glaucoma. A first tube implanted in the anterior chamber of the eye has an end covered with a thin membrane having a small hole in the center which can be enlarged by using an ophthalmic laser. A second tube is interconnected with the first tube at a right angle to form a single T-shaped shunt with the second tube implanted in the lumen of Schlemm's Canal. A third tube, with a porous end plate, is implanted into the subconjunctival-tenon space and is connected to the first and second tubes to form the “cross-shaped” double shunt. The end plate has small pores that control the rate of aqueous humor outflow. The first T-shaped shunt can be used as an independent single-shunting device by omitting the addition of the third tube.

### BACKGROUND OF THE INVENTION

1. Field of the Invention

This invention relates generally to the drainage of aqueous humor from an eye to relieve the elevated pressure characteristic of glaucoma, and in particular, to a double shunt, using a cross-shaped tubular structure for bypassing aqueous humor out of the anterior chamber of the eye into both Schlemm's Canal and the subconjunctival-tenon space utilizing a membrane and post-implant laser fabricated changeable opening for controlling the flow of aqueous humor in response to pressure.

2. Description of the Prior Art

Aqueous humor is continuously produced by the ciliary body in the posterior chamber of the eye, and from there it flows through the pupil into the anterior chamber of the eye. In order to maintain relatively constant intraocular pressure, aqueous humor must be drained away continuously. It's path passes primarily through the trabecular meshwork of the anterior chamber and into the canal of Schlemm, before draining into the veins which leave the eye.

The relevant structures of the eye and some of their functions will be briefly described to provide an understanding of the present invention. The sclera is the thick collagenous membrane which forms the outer walls of the almost ellipsoidal eye, except at the anterior central median, where the thin optical membrane, known as the cornea, forms a window into the eye. The sclera deforms and responds to extraocular muscle movements and ocular pressure changes that occur during normal physiological functions of the eye. The junction where the cornea and sclera merge is referred to as the limbus. The conjunctiva is a membrane of tissue which extends posteriorly from the limbus over the front portion of the sclera and then projects in a forward direction lining the upper and lower eyelids. Tenon's capsule is a thin tissue membrane that attaches anteriorly at the limbus beneath the conjunctiva and extends posteriorly over the sclera and extraocular muscles to attach at the back of the bony orbit within which the eye sits.

The ciliary body begins internally in the eye at the limbus and extends along the interior of the sclera. The iris, which extends radially into the center void of the eye from the anterior section of the ciliary body, forms the edges of the central space known as the pupil and acts as a diaphragm controlling the size of the pupil. The anterior chamber of the eye, which is bound anteriorly by the cornea and posteriorly by the iris, is filled with a fluid produced primarily by the ciliary body, said fluid being referred to as the aqueous humor. The aqueous humor passes through the Trabecular meshwork into the canal of Schlemm, and from Schlemm's canal the aqueous humor is subsequently drained into the veins leaving the eye.

The intraocular pressure of the eye is produced by the amount of aqueous humor within the eye at any given time and, in a normal and healthy eye, is maintained at a constant level by natural bodily functions which balance the production and outflow of the aqueous humor. The disease state of glaucoma is most often characterized by an abnormally elevated intraocular pressure from decreased outflow of the aqueous humor.

Normal intraocular pressure is typically about 15.+−0.4 mm Hg, but may rise to 21 mm Hg. Pressures within the eye that are substantially above this range are considered abnormally high. Chronically elevated intraocular pressure (IOP—resulting, for example, from a defect in intraocular drainage) can give rise to glaucoma. When the aqueous humor excretory pathway is blocked, the aqueous humor cannot pass out of the eyeball at an adequate rate, the IOP rises, the eyeball becomes harder, and the optic nerve atrophies by the pressure applied on its fibers leaving the retina. A characteristic optic neuropathy develops, resulting in progressive death of the ganglion cells in the retina, restriction of the visual field, and eventual blindness. Advanced stages of the disease are characterized also by significant pain. Glaucoma can cause irreversible damage to certain structures of the eye, including the optic nerve, and is a leading cause of blindness in the United States.

Throughout the United States, Europe and most of the first world countries, glaucoma is the most prevalent sight-threatening disease and is, on a world wide basis, responsible for approximately ten percent of all blindness.

Glaucoma is a significant healthcare problem with immediate and long term ramifications, both physical and financial. Glaucoma is the leading cause of blindness in the United States, where over 300,000 new cases are reported each year. In the U.S. more than 95,000 glaucoma patients lose some degree of sight each year due to the disease, with greater than 5,500 experiencing total blindness. As treatment costs on a per year basis are estimated to be $1.5 billion, based upon more than two million annual office visits, the socioeconomic impact of glaucoma is significant.

The causes of glaucoma are poorly understood; however, vasomotor and emotional instability, hyperopia, and especially heredity are among the recognized predisposing factors. Along with persons predisposed to glaucoma by virtue of family history, individuals at higher risk of developing glaucoma are those 35 years of age or older or those with diabetes or positive glucose tolerance tests. The disease also strikes African-Americans in disproportionate numbers. They are four to five times more likely to develop glaucoma and are up to six times more apt to suffer a complete loss of sight.

There are many types and causes of glaucoma. Treatment of the disease depends on both the patient and the form of glaucoma. As a rule, the damage caused by glaucoma can not be reversed. The goal, therefore, of glaucoma treatment is to prevent further damage and to preserve existing vision.

Glaucoma can often be controlled with medical therapy, typically through topical medications, such as pilocarpine, timolol maleate, betaxolol, or epinephrine, and also through systemic medications, including acetazolamide. Medical therapy either decreases the rate of production of aqueous humor, or increases its outflow from the anterior chamber. However, with many patients these procedures are not effective because the patients fail to follow the treatment prescribed, due to either negligence or the relatively high cost of the medication. Other potential problems with medical treatment include side effects and inadequate control of the intraocular pressure.

If the maximum-tolerated dose of medication fails to control the intraocular pressure, then laser trabeculoplasty or filtering surgery to increase aqueous drainage is usually indicated. These procedures seek to increase the rate of outflow of aqueous humor. An iridectomy, removal of a portion of the iris, is often used in angle-closure glaucoma wherein there is an occlusion of the trabecular meshwork by iris contact. Removal of a piece of the iris then gives the aqueous free passage from the posterior to the anterior chambers in the eye. A trabeculotomy, opening the inner wall of Schlemm's canal, is often performed in cases of open-angle glaucoma so as to increase the outflow of the aqueous, thereby decreasing intraocular pressure. While often successful, these surgical techniques possess inherent risks associated with invasive surgery on an already afflicted eye. Furthermore, the tissue of the eye can grow back to the pre-operative condition, thereby necessitating the need for further treatment.

Other types of surgical procedures seek to reduce the formation of aqueous humor, by destroying the tissue where it is created. These procedures are typically indicated only after filtering surgery has failed. If such filtering surgery has failed to control the intraocular pressure, or if the patient has a poor prognosis for filtering surgery, implantation of a glaucoma shunt may be indicated.

Glaucoma shunts typically drain aqueous humor from the anterior chamber of the eye to the fibrous capsule (bleb) which forms around a collecting device placed on the posterior portion of the globe of the eye, and the humor is then reabsorbed into the vascular system. The bleb is formed apparently due to an immune response against the shunt, which the host recognizes as a foreign body. Bleb formation is essential for a successful implant procedure and recovery by the patient.

Glaucoma shunts typically consist of a silicone elastomer catheter which is inserted into the anterior chamber, and which connects to an episcleral plate or an encircling band. Episcleral plates are commonly made of silicone elastomer, polypropylene or acrylic materials.

While there are many shunts for draining aqueous fluid to relieve intraocular pressure, none provide a shunt from the anterior chamber to Schlemm's canal or provide a double-shunting device.

U.S. Pat. No. 5,346,464, issued Sep. 13, 1994 to Camras, claims an apparatus for reducing intraocular pressure includes first and second resilient flexible tubes connected together to permit fluid flow therethrough. The first tube has one end inserted within the anterior chamber of the eye to drain fluid therefrom and extends through an aperture in the conjunctival layer. The second tube is connected to the external end of the first tube, and has an operable valve at the free end thereof which opens when subjected to a predetermined fluid pressure, to thereby reduce the intraocular pressure of the eye. A filter is mounted within the second tube to prevent bacteria from entering the anterior chamber of the eye, while permitting replacement of the filter as desired. A method for reducing intraocular pressure includes the step of inserting a first end of the first described tube into the anterior chamber of the eye, and positioning the second end external to the ocular surface of the eye. The second end of the tube is passed through an aperture in the conjunctival layer, so as to be positioned external to the ocular surface of the eye. The second tube is then connected to the first tube with the operable valve preferably located in the conjunctival cul-de-sac.

U.S. Pat. No. 5,626,558, issued May 6, 1997 to Suson, provides an adjustable flow rate implantable shunt device for use in treating glaucoma. The device comprises a tubular member having a first sealed end and an open second end which are joined by a wall. The first end is inserted into the anterior portion of the eye for receipt of aqueous humor, and the second end is fastened to the sclera of the eye. The first end of the shunt device is sealed when implanted, so that flow of aqueous humor through the device immediately after implantation is prevented. After a sufficient post-implantation period such that a fibrous capsule has formed around the shunt device, at least one perforation is made along the segment of the wall of the tubular member which is located within the anterior portion of the eye to allow aqueous humor to flow through and out of the device, thereby lowering intraocular pressure within the eye. The flow rate of aqueous humor through the device can be adjusted periodically by placing additional perforations along the portion of the tubular member located within the anterior chamber of the eye. The perforations are conveniently made using a laser.

U.S. Pat. No. 5,433,701, issued Jul. 18, 1995 to Rubinstein, shows an apparatus for reducing pressure in an anterior chamber of an eye. The apparatus includes an anterior portion configured for implantation through a scleral tunnel such that a leading edge thereof is within the anterior chamber. A plurality of channels are defined through the anterior portion, the channels being open to an external environment of the anterior portion to provide fluid communication between the anterior chamber of the eye and the channels. The apparatus further includes a body portion extending from the anterior portion distal the leading edge of the anterior portion of the apparatus. The body portion is configured for implantation between conjunctival and scleral tissues of the eye. The body portion defines a channel therethrough, the channel being in fluid communication with one or more of the plurality of channels formed through the anterior portion of the apparatus. Occlusion means are disposed in one or more of the channels formed through the anterior portion of the apparatus. Each of the occlusion means has a first position in which flow through the channels formed through the anterior portion is obstructed and a second position in which flow through the channels formed through the anterior portion is not obstructed. The occlusion means is adjustable between the first position and the second position to provide selective control of the flow of aqueous through the apparatus.

U.S. Pat. No. 5,178,604, issued Jan. 12, 1993 to Baerveldt, describes an implant for use in the treatment of glaucoma wherein the implant comprises an elastomeric plate having a non-valved elastomeric drainage tube attached thereto. The plate is elliptical in shape and curved so as to conform to the curvature of the eye. The plate is inserted into the eye in an incision made in the Tenon's capsule and sutured to the sclera. The drainage tube is tunnelled through the Tenon's capsule and cornea and inserted into the anterior chamber, thus providing patent fluid communication between the anterior chamber and the elastomeric plate. The flexible structure of the plate allows the plate to be easily inserted, thus reducing the surgical procedure length. In addition, the pliable material minimizes the risk of damage and trauma to surrounding tissues in the insertion process.

U.S. Pat. No. 5,300,020, issued Apr. 5, 1994 to L'Esperance, Jr., discloses a surgically implantable device for controlled drainage flow of aqueous fluid from the anterior chamber of the eye into nearby subconjunctival space, all in relief of a glaucomatous condition of excessive pressure within the eye. The device includes provision for so controlling the rate of aqueous flow as to assure against anterior-chamber collapse, thus avoiding irreparable damage which might otherwise result to the corneal endothelium, to the iris, or to the lens of the eye.

U.S. Pat. No. 5,073,163, issued Dec 17, 1991 to Lippman, indicates an apparatus for treating glaucoma which can be mounted directly onto the outer surface of the eyeball with a portion of the apparatus to connect with the interior of the eyeball. The apparatus includes a plastic block including a plurality of tiny through openings. Liquid from the interior of the eyeball is to seep through the openings in the plastic block to relieve excess pressure within the eyeball. This leakage is to occur only when the pressure level within the eyeball exceeds a predetermined level.

U.S. Pat. No. 5,486,165, issued Jan. 23, 1996 to Stegmann, puts forth a method and an appliance for carrying out the method, by means of which the necessary outflow of the aqueous humour which is continuously being renewed in the eye is ensured, and thus the natural intraocular pressure is maintained, are proposed. For the treatment, the sclera (13) undergoes operative lamellar incision for partial exposure of the canal of Schlemm (15), and the portion (13′) which is opened out is held by means which are not depicted. A medium is introduced into the canal of Schlemm (15) by means of a tube (20) which is introduced into the circular canal of Schlemm (15), by which means the upstream trabecular tissue (15′) is hydraulically expanded and traumatically opened at several points (15″) and, at the same time, the points (15″) are wetted by the highly viscous medium.

U.S. Pat. No. 3,159,161, issued Dec. 1, 1964 to Ness, concerns a fistulizing canaliculus for drainage of aqueous humor from the anterior chamber of the eye out through a tube to an orthogonal flange with cross-shaped grooves to facilitate outflow into the retrobulbar space.

While there are many shunts to relieve intraocular pressure related to glaucoma, none provide a double shunting device with a sized opening to control flow relative to the pressure buildup.

### SUMMARY OF THE INVENTION

An object of the present invention is to provide a relief for uncontrolled glaucoma by offering a shunt to facilitate the outflow of aqueous humor from the eye.

Another object of the present invention is to provide an ocular shunt that feeds into Schlemm's canal from the anterior chamber of the eye for an unobstructed positive outflow of fluid.

One more object of the present invention is to provide a double ocular shunt to drain fluid from the anterior chamber of the eye into both Schlemm's canal and the subconjunctival-tenon space.

A corollary object of the present invention is to provide a third tube, with a porous end plate, attached to or formed as an extension of the T-shaped ocular shunt to create a double shunt which is cross-shaped, with the end plate having small pores that control the rate of outflow of aqueous humor to prevent an overflow of too much fluid from the eye.

An additional object of the present invention is to provide an ocular shunt with a membrane having a post-implant laser fabricated changeable opening for controlling flow in response to pressure to prevent too much buildup of pressure in the eye as well as stopping flow when the pressure level is too low.

An associated object of the present invention is to provide an ocular shunt which bypasses the trabecular meshwork complex, which is the major resistance to outflow of aqueous humor.

A further object of the present invention is to provide a device made of biocompatible materials such as silicone and acrylic.

In brief, a device for controlling intraocular pressure (IOP) by implanting a “cross-shaped” tubular structure for shunting aqueous humor out of the eye has a first tube implanted in the anterior chamber of the eye where the aqueous humor is located. The opening in the anterior chamber end of the first tube is covered with a thin membrane which has a small hole in the center. The small hole can be enlarged by using an ophthalmic laser.

A second tube, which may be formed in one piece with the first tube, connects to the opposite end of the first tube, with the first tube intersecting the second tube at a centerpoint of the second tube, to form a T-shaped first shunt. The two ends of the second tube are implanted in the lumen of Schlemm's Canal. The first shunt bypasses the trabecular meshwork complex, which is the major resistance to outflow of aqueous humor and shunts intraocular fluid into the Schlemm's Canal, where shunted fluid drains out of the eye through natural channels (direct and indirect aqueous veins and episcleral vein) and returns the fluid to blood circulation.

A third tube, with a porous end plate, is implanted into the subconjunctival-tenon space and connected to the first and second tubes at the intersection of the first and second tubes to form a “cross-shaped” second shunt. The second shunt bypasses the natural draining structure and shunts aqueous humor out of the eye into subconjunctival-tenon space where the aqueous humor diffuses into connective tissue. The end plate has small pores that control the rate of outflow to prevent overflow. The first T-shaped shunt can be used as an independent single-shunting device by omitting the addition of the third tube.

An advantage of the present invention is to provide an immediate relief for uncontrolled glaucoma.

Another advantage of the present invention is to provide an ocular shunt implanted into the lumen of Schlemm's canal for an unobstructed positive outflow of fluid.

An additional advantage of the present invention is to provide a double shunt that is capable of drawing forth more aqueous fluid than a single shunt.

One more advantage of the present invention is that it regulates the intake flow of aqueous fluid by the size of the membrane hole.

A related advantage of the present invention is that you can create or enlarge the membrane hole after the ocular shunt is implanted using an ophthalmic laser, the size of which hole will regulate fluid flow affecting ocular pressure.

Yet another advantage of the present invention is that it regulates the output flow of aqueous fluid by a network of small pores found at the endplate of the third tube.

An extra advantage of the present invention is the ability to modify the number of tubes implanted to result in the desired therapy.

Still another advantage of the present invention is to provide a device made of biocompatible materials such as silicone and acrylic thereby insuring that the shunt will not be rejected.

### BRIEF DESCRIPTION OF THE DRAWINGS

These and other details of my invention will be described in connection with the accompanying drawings, which are furnished only by way of illustration and not in limitation of the invention, and in which drawings:

FIG. 1 is a perspective view of the combined dual shunt in place in the eye;

FIG. 2 is a cross-sectional view taken through the dual shunt of FIG. 1;

FIG. 3 is a diagrammatic view of the combined dual shunt of FIG. 1 in place in the eye showing the eye structure;

FIG. 4 is a cross-sectional view taken through the T-shaped shunt which drains aqueous fluid from the anterior chamber of the eye to Schlemm's canal.

### BEST MODE FOR CARRYING OUT THE INVENTION

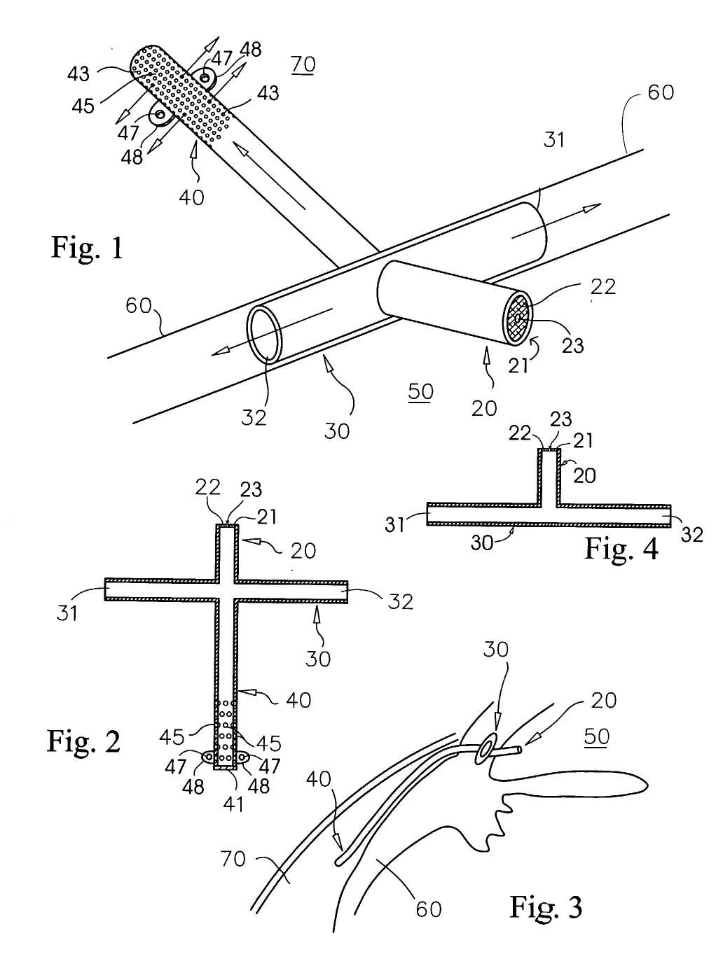

In FIGS. 1-4, an ocular shunt 20, 30, 40 is shown for draining aqueous fluid from an anterior chamber 50 of the eye to relieve pressure buildup from glaucoma. The shunt has a first tube 20 with an interior first lumen therethrough, a distal opening at one end, and a proximal opening at another end. Both openings communicate with the first lumen. The proximal end has a membrane 22 covering the opening 21. The membrane is pierced by a membrane hole 23 therethrough to admit fluid through the membrane hole 23. The proximal end is capable of being inserted into an anterior chamber 50 of an eye and is also capable of receiving a controlled flow of aqueous fluid through the first tube 20. The flow of aqueous fluid is controlled by the size of the membrane hole 23. The membrane hole 23 may be created and enlarged by an ophthalmic laser with the ocular shunt in place in the eye.

In FIGS. 1, 2 and 4, a second tube 30 attaches transversely to the first tube 20. The second tube 30 has a second lumen therethrough and two open ends 31, 32 which communicate with the second lumen. The second lumen further communicates with the first lumen from the first tube 20. The second tube 30 is capable of being inserted into Schlemm's canal 60 of the eye so that ocular fluid flows from the anterior chamber 50 of the eye through the first and second lumens and out of the two open ends 31 and 32 of the second tube 30 into Schlemm's canal 60. In FIG. 4 a T-shaped shunt is used for draining aqueous fluid from the anterior chamber 50 through the tube 20 of the eye to Schlemm's canal 60 out of the ends 31 and 32 of the second tube 30, as shown in FIG. 1.

In FIGS. 1-4, the first tube 20 and the second tube 30, and third tube 40 are formed of molded biocompatible material.

In FIGS. 1-3, a third tube 40 is attached to the first 20 and second 30 tubes at an intersection of the first 20 and second 30 tubes. The third tube 40 has a third lumen, which communicates with at least the first lumen of the first tube 20, and an attaching end connected to the first 20 and second 30 tubes and also a plate end 45 terminating in a porous network of openings. Both the attaching end and the plate end 45 communicate with the third lumen. The plate end 45 is capable of being inserted into the subconjunctival-tenon space 70 of the eye so that aqueous fluid further flows from the anterior chamber 50 through the first 20 and third tubes 40 into the subconjunctival-tenon space 70 using the porous network of openings 43. The plate end 45 of the third tube 40 is secured in place by sutures through openings 47 in side tabs 48 of the third tube 40.

In FIGS. 1, 2 there is a network of openings comprised of a series of pores 43 through the plate end 45 of the third tube 40. The pores 43 are sized to control the outflow rate of aqueous fluid through the pores 43.

It is understood that the preceding description is given merely by way of illustration and not in limitation of the invention and that various modifications may be made thereto without departing from the spirit of the invention as claimed.

What is claimed is:

1. An ocular shunt for draining aqueous fluid from an anterior chamber of the eye to relieve pressure buildup from glaucoma, the shunt comprising:
a first tube having an interior first lumen therethrough and a distal opening at one end and a proximal opening at another end both openings communicating with the first lumen, the proximal end having a membrane covering the opening and the membrane having a membrane hole therethrough to admit fluid through the membrane hole, the proximal end capable of being inserted in an anterior chamber of an eye and capable of receiving a controlled flow of aqueous fluid through the first tube, the flow being controlled by the size of the membrane hole;
a second tube attached transversely to the first tube, the second tube having a second lumen therethrough and two open ends communicating with the second lumen, the second lumen further communicating with the first lumen from the first tube, the second tube capable of being inserted in Schlemm's canal of the eye so that ocular fluid flows from the anterior chamber of the eye through the first and second lumens and out the two open ends of the second tube into Schlemm's canal.

2. The ocular shunt of claim 1 wherein the first tube and the second tube are formed together of molded biocompatible material.

3. The ocular shunt of claim 1 further comprising a third tube attached to the first and second tubes at an intersection of the first and second tubes, the third tube having a third lumen communicating with at least the first lumen of the first tube and an attaching end connected to the first and second tubes and a plate end terminating in a porous network of openings, both the attaching end and the plate end communicating with the third lumen, the plate end capable of being inserted in the subconjunctival-tenon space of the eye so that aqueous fluid further flows from the anterior chamber through the first tube and the third tube into the subconjunctival-tenon space through the porous network of openings.

4. The ocular shunt of claim 3 wherein the first tube, the second tube, and the third tube are formed together of molded biocompatible material.

5. The ocular shunt of claim 3 wherein the network of openings is comprised of a series of pores through the plate end which pores are sized to control the rate of outflow of aqueous fluid through the pores.

6. The ocular shunt of claim 1 wherein the membrane hole is capable of being created and capable of being enlarged by an ophthalmic laser means with the ocular shunt in place in the eye.
